(19) United States
(12) Patent Application Publication
(10) Pub. No.: US 2022/0047494 A1
(43) Pub. Date: Feb. 17, 2022

(54) ANTIBACTERIAL, DEODORIZATION, EASING OF ACNE AND ATOPY OR SKIN CALMING-FUNCTIONAL COMPOSITION FOR Y-ZONE CLEANLINESS CONTAINING QUERCUS ACUTISSIMA SAP AND ZINC RICINOLEATE

(71) Applicant: MEDICLASSIC.CO.LTD, Namyangju-si (KR)

(72) Inventors: Mi Kyung LEE, Seoul (KR); Ji Woo KIM, Seoul (KR)

(73) Assignee: MEDICLASSIC.CO.LTD, Namyangju-si (KR)

(21) Appl. No.: 17/396,766

(22) Filed: Aug. 9, 2021

(30) Foreign Application Priority Data
Aug. 12, 2020 (KR) 10-2020-0100907

(51) Int. Cl. A61K 8/9789; A61K 8/27; A61K 8/46; A61K 8/41; A61K 8/365; A61K 8/42; A61K 8/04; A61Q 19/10; A61Q 17/00

(52) U.S. Cl. CPC A61K 8/9789; A61K 8/27; A61K 8/463; A61K 8/41; A61Q 17/005; A61K 8/42; A61K 8/046; A61Q 19/10; A61K 8/365

(57) ABSTRACT

The present invention relates to an antibacterial, deodorization, easing of acne and atopic dermatitis, or skin calming-functional composition for Y-zone cleanliness containing Quercus acutissima sap, wherein the composition for Y-zone cleanliness is a composition used for cleaning a Y zone of women, which contains Quercus acutissima sap to provide antibacterial and deodorizing effects and to alleviate acne and atopic dermatitis or to provide a skin-calming function.

### BACKGROUND OF THE INVENTION

### 1. Field of the Invention

The present invention relates to an antibacterial, deodorization, easing of acne and atopic dermatitis, or skin calming-functional composition for Y-zone cleanliness containing Quercus acutissima sap.

Specifically, the present invention relates to a composition used for cleaning a Y zone of women, which contains Quercus acutissima sap to provide antibacterial and deodorizing effects and to alleviate acne and atopic dermatitis or to provide a skin-calming function.

### 2. Description of the Related Art

A female genitourinary system includes a uterus, vagina, vulva, urethra, etc., and is easily exposed to external infections due to its anatomical, physiological and functional characteristics. For example, since the vagina is rich in nutrients and is located deep in the human body, a temperature and humidity are high, and thus the vagina is easily invaded by various strains, including bacteria or fungi, which are the main causes of vaginitis, and various diseases are highly likely to occur. Thus, it is desirable to always keep the vagina clean and not to expose the vagina to bacterial infection from outside with care.

In addition, a female reproductive system may be easily infected with germs through a mucous membrane during sexual life. Furthermore, if a contraceptive device is placed in the uterus for a long period of time, chronic inflammation may be induced, and the mucous membrane of the corresponding body part may be often damaged when a medical device is introduced into the body or when a mammal is delivered. The damage may increase the chances of infection by pathogens and cause inflammation.

For women, inflammation of the genitourinary system is one of the most common diseases among patients who visit a gynecologist, and this inflammation occurs due to very diverse causes ranging from bacteria to parasites and viruses. These patients suffer from accompanying pruritus, severe inflammation, and bad odor. In particular, human papillomavirus infection is very closely related to cervical cancer, thus requiring close attention. Thus, it is very important to reduce fatal complications through appropriate prevention, diagnosis and treatment.

Representative infections of the female genital organs (vulva, vagina, cervix, etc.) include bacterial vaginosis, trichomonas vaginitis, vulvar and vaginal candidiasis, chronic vulvar candidiasis, general inflammatory vaginosis, atrophic vaginitis, cervicovaginitis, etc. In addition, when a large amount of bleeding is accompanied before and after menstruation, a very uncomfortable feel may be caused in terms of hygiene, along with an increasing chance of infection.

In order to prevent the above female diseases and remove unpleasant odors, vaginal cleaners (cleaning agents), vulvar cleaners, etc., are often used. As a cleansing agent for genital cleanliness that is generally used to prevent vaginitis, a transparent or lotion type prepared by sulphonating fatty acids, distilled lauryl alcohol, or the like, and containing emollients, thickeners, nutrients, foaming agents, organic disinfectants, etc., has been released on the market. In addition, there may be an example in which a surfactant having detergency and/or povidone-iodine, a drug medicine is contained as a main ingredient. Povidone-iodine, which is a disinfectant prepared by the chemical synthesis mentioned above, is known to be effective in the prevention and treatment of candida vaginitis, trichomonas vaginitis, and other vaginal infections, but has a risk of causing hypersensitivity to iodine, etc., or pruritus, burning sensation, skin ulcer, contact dermatitis, etc., due to mucosal injury and may cause hypothyroidism by increasing a level of inorganic iodine in the vagina or total iodine in the blood. In addition, healthy women have a vaginal acidity maintained at pH 3.2 through the secretion of lactic acid by beneficial microorganisms in the vagina. Considering that most harmful bacteria are inhibited or killed at this low acidity, a buffer solution formulation of pH 3.5 with lactic acid as a main ingredient and with the addition of lactoserum has been proposed, but the formulation is merely an intravaginal titratable acidity maintainer that maintains a vaginal pH in a normal state, and is hardly expected to provide a fundamental treatment.

In addition, for the prevention or treatment of these diseases, vaginal application preparations containing an active ingredient such as antibiotics, etc., are used in the vaginal tablet, lotion, cream, ointment, and liquid types. The use of such general sanitizers, disinfectants, antibiotics, etc., not only increases the possibility of the occurrence of antibiotic-resistant microorganisms, but also indiscriminately attacks normal lactobacillus, etc., in the vagina, which have a self-cleaning effect by maintaining a proper acidity of the secretions in the vagina, and thus remove even useful microorganisms therein to easily replace the same with pathogenic anaerobic bacteria, etc., thereby hardly providing a complete recovery along with many side effects and leading to a frequent recurrence. Accordingly, it is necessary to develop a method capable of compensate the above problems.

Meanwhile, in relation to the conventional cleaning agent composition for female genital cleanliness, Korean Unexamined Patent Publication No. 10-2006-0036168 titled “Vaginal cleansing agent composition comprising pomegranate bark extract and a method for preparing the same” discloses a vaginal cleaning agent composition containing vegetable ingredients such as pomegranate bark extract, etc., suitable for sensitive skin with an excellent cleansing effect and a method for preparing the same, and Korean Registered Patent No. 10-0548153 titled “Method of extracting an effective ingredient useful for the prevention and treatment of vaginitis and a cleaning agent composition comprising an extract extracted by using the same” discloses a cleansing agent composition for preventing and treating vaginosis, containing an extract of a herbal medicinal mixture consisting of licorice, rehmannia root, peony, cnidium, angelica, Torilis Fructus, lycium root and Angelica dahurica as an effective ingredient.

In addition to the conventional compositions as described above, various types of cleaning agent compositions for female genital cleanliness have been disclosed, but there is a continuous need for a novel composition for female Y-zone cleanliness containing natural substances with antibacterial properties as a main ingredient as well as various functions such as human-friendliness, antibacterial action, deodorization, acne and atopic relief, skin soothing or the like.

### RELATED ART DOCUMENTS

### Patent Documents

(Patent Document 1) Korean Unexamined Patent Publication No. 10-2006-0036168

(Patent Document 2) Korean Registered Patent Publication No. 10-0548153

### SUMMARY OF THE INVENTION

The present invention may provide a composition for Y-zone cleanliness which contains Quercus acutissima sap and an extract of Quercus acutissima bark as a human-friendly natural substance, so as to provide antibacterial and acne and atopy-alleviating functions, and also contains zinc ricinoleate to provide an excellent deodorizing effect.

The present invention may provide a composition for Y-zone cleanliness, which inhibits the growth of Staphylococcus epidermidis strain, a secondary infection bacillus, as well as a first infection bacillus, and also inhibits the growth of Candida albicans (ATCC 10231), a yeast that causes inflammatory or bacterial skin diseases, so as to provide antibacterial and acne and atopy-alleviating effects, and also contains an intensive moisturizing ingredient to provide a skin-calming effect.

The present invention may also provide a mist containing the composition for Y-zone cleanliness.

The present invention may further provide a method for preparing the composition for Y-zone cleanliness.

The present invention, which is applied to solve the aforementioned problems, relates to a composition for Y-zone cleanliness, a mist containing the same, and a method for preparing the composition for Y-zone cleanliness.

The composition for Y-zone cleanliness may be a mixture of 70 to 90 wt % of a first phase ingredient (A) and 10 to 30 wt % of a second phase ingredient (B). In addition, the first phase ingredient (A) may include 40 to 60 parts by weight of Quercus acutissima sap, 20 to 40 parts by weight of an ethyl acetate fraction of high-pressure and ultrasonic extract of Quercus acutissima bark, 0.05 to 2 parts by weight of water-phase zinc gluconate, 0.1 to 3 parts by weight of Zanthoxylum piperitum fruit extract, 0.1 to 3 parts by weight of Pulsatilla koreana flower extract, and 0.1 to 3 parts by weight of Usnea barbata extract, and the second phase ingredient (B) may include 40 to 60 parts by weight of zinc ricinoleate, 30 to 40 parts by weight of laureth-3, 10 to 20 parts by weight of tetrahydroxypropylethylenediamine, and 1 to 10 parts by weight of propylene glycol.

In one example, the Quercus acutissima sap may be prepared through the steps of putting Quercus acutissima wood chips into a drying furnace of which an outside is captured by a steam tank and drying to produce Quercus acutissima sap vapor, and collecting condensed water produced by bringing the Quercus acutissima sap vapor into contact with a cooling coil in a cooling tank.

In one example, the first phase ingredient (A) may include 50 to 60 parts by weight of Quercus acutissima sap; and 25 to 35 parts by weight of an ethyl acetate fraction of high-pressure and ultrasonic extract of Quercus acutissima bark.

In one example, the ethyl acetate fraction of high-pressure and ultrasonic extract of Quercus acutissima bark may be prepared through the steps of: washing and pulverizing Quercus acutissima bark, followed by drying; preparing a sample by mixing the dried Quercus acutissima bark with distilled water, and carrying out a low-temperature and high-pressure extraction with respect to the sample for 5 to 15 minutes at a low temperature of 20° C. to 30° C. and under a pressure of 4,000 bar to 6,000 bar by using a high-pressure extraction device; carrying out an ultrasonic extraction with respect to the sample that has been subject to the low-temperature and high-pressure extraction for 30 to 90 minutes at a frequency of 40 kHz to 70 kHz by using an ultrasonic extractor; carrying out a hot water extraction with respect to the sample that has been subject to the ultrasonic extraction with distilled water for 10 to 20 hours at a temperature of 40° C. to 60° C. so as to obtain a high-pressure and ultrasonic extract of Quercus acutissima bark; filtering and concentrating the high-pressure and ultrasonic extract, followed by free-drying and pulverization; and dispersing the resulting powdered high-pressure and ultrasonic extract in distilled water, and then carrying out a solvent fractionation using ethyl acetate so as to obtain an ethyl acetate fraction of high-pressure and ultrasonic extract of Quercus acutissima bark.

In one example, the water-phase zinc gluconate may contain purified water as a main ingredient and may include a zinc salt of gluconic acid represented by formula 1 below within a range of 50,000 ppm to 100,000 ppm.

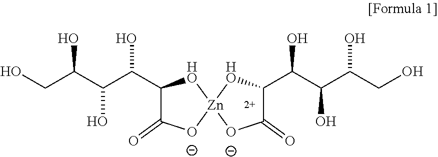

In one example, the Zanthoxylum piperitum fruit extract, the Pulsatilla koreana flower extract, and the Usnea barbata extract may each include an ultrasonic and low temperature water extract prepared by performing an ultrasonic pretreatment with Zanthoxylum piperitum fruit, Pulsatilla koreana flower and gingko leaves by using an ultrasonic extractor for 30 to 60 minutes under a condition of 100 kHz to 200 kHz, and then carrying out a low-temperature water extraction in a temperature range of 20° C. to 40° C. for 6 to 24 hours.

In one example, the second phase ingredient (B) may further include 0.5 to 2 parts by weight of panthenol.

The present invention also relates to a mist for Y-zone cleanliness containing the composition for Y-zone cleanliness.

The present invention also relates to a method for preparing the composition for Y-zone cleanliness. The method for preparing the composition for Y-zone cleanliness may include the steps of: preparing a first phase ingredient (A); preparing a second phase ingredient (B); and mixing the first phase ingredient (A) and the second phase ingredient (B) at a mixing ratio of 7:3 to 9:1(A:B).

The step of preparing the first phase ingredient (A) may include the steps of: putting Quercus acutissima wood chips into a drying furnace of which an outside is captured by a steam tank and drying to produce Quercus acutissima sap vapor, and collecting condensed water produced by bringing the Quercus acutissima sap vapor into contact with a cooling coil in a cooling tank; sequentially carrying out a high-pressure and ultrasonic extraction with regard to a sample including the dried Quercus acutissima bark after washing by using a high-pressure extraction device and an ultrasonic extractor, and then carrying out a solvent fractionation with respect to a freeze-dried product of the high-pressure and ultrasonic extract so as to obtain an ethyl acetate fraction of high-pressure and ultrasonic extract of Quercus acutissima bark; and mixing 40 to 60 parts by weight of Quercus acutissima sap, 20 to 40 parts by weight of an ethyl acetate fraction of high-pressure and ultrasonic extract of Quercus acutissima bark, 0.02 to 2 parts by weight of water-phase zinc gluconate, 0.1 to 3 parts by weight of Zanthoxylum piperitum fruit extract, 0.1 to 3 parts by weight of Pulsatilla koreana flower extract, and 0.1 to 3 parts by weight of Usnea barbata extract.

The step of preparing the second phase ingredient (B) may include mixing 40 to 60 parts by weight of zinc ricinoleate, 30 to 40 parts by weight of laureth-3, 10 to 20 parts by weight of tetrahydroxypropylethylenediamine, and 1 to 10 parts by weight of propylene glycol.

In one example, the step of obtaining an ethyl acetate fraction of high-pressure and ultrasonic extract of Quercus acutissima bark may include the steps of: washing and pulverizing Quercus acutissima bark, followed by drying; preparing a sample by mixing the dried Quercus acutissima bark with distilled water, and carrying out a low-temperature and high-pressure extraction with respect to the sample for 5 to 15 minutes at a low temperature of 20° C. to 30° C. and under a pressure of 4,000 bar to 6,000 bar by using a high-pressure extraction device; carrying out an ultrasonic extraction with respect to the sample that has been subject to the low-temperature and high-pressure extraction for 30 to 90 minutes at a frequency of 40 kHz to 70 kHz by using an ultrasonic extractor; carrying out a hot water extraction with respect to the sample that has been subject to the ultrasonic extraction with distilled water for 10 to 20 hours at a temperature of 40° C. to 60° C. so as to obtain a high-pressure and ultrasonic extract of Quercus acutissima bark; filtering and concentrating the high-pressure and ultrasonic extract, followed by free-drying and pulverization; and dispersing the resulting powdered high-pressure and ultrasonic extract in distilled water, and then carrying out a solvent fractionation using ethyl acetate so as to obtain an ethyl acetate fraction of high-pressure and ultrasonic extract of Quercus acutissima bark.

A composition for Y-zone cleanliness according to the present invention can contain a natural ingredient obtained from Quercus acutissima to provide antibacterial and acne and atopy-alleviating effects in a skin-friendly way without side effects to the human body.

The composition for Y-zone cleanliness according to the present invention can also contain an intensive moisturizing ingredient to provide a skin-calming effect, and further contain zinc ricinoleate to provide an excellent deodorizing effect.

Of course, the effect of the present invention is not limited within the above-mentioned range.

### BRIEF DESCRIPTION OF THE DRAWINGS

FIG. 1 shows the results of a cytotoxicity experiment on Quercus acutissima sap and Quercus acutissima bark extract and fraction according to the present invention.

FIGS. 2 and 3 show the results of an experiment on antibacterial activity with respect to Quercus acutissima sap and an extract and fraction of Quercus acutissima bark according to the present invention.

### DETAILED DESCRIPTION OF THE INVENTION

Hereinafter, the present invention will be described in more detail with reference to drawings and examples.

In the present specification, the terms of a singular form may include plural forms unless otherwise specified.

For the terms used in the present specification, currently widely used general terms have been selected with the consideration of the functions in the present invention, but may vary depending on a skilled person's intention or precedent in the art, the emergence of new technology, and the like. In addition, in a specific case, there is a term arbitrarily selected by the applicant, and in this case, the meaning will be described in detail in the description of the corresponding invention. Thus, the term used in the present invention should be defined based on the meaning of the term and the overall content of the present invention, rather than the name of a simple term.

The exemplary embodiments of the present invention may be applied with various modifications and have various embodiments, but specific embodiments are illustrated in the drawings and described in detail. However, this is not intended to limit the present invention to the specific embodiments, but it should be understood to include all modifications, equivalents and substitutes in the spirit and scope of the present invention. In the description of the embodiments, a detailed description of known arts incorporated herein will be omitted when it may make the subject matter of the present invention unnecessarily unclear.

In the present specification, the terms “comprise,” “include,” “contain” or the like are intended to designate that the features, the numbers, the steps, the operations, the components, the ingredients, the parts or combinations thereof are present, and are not to be understood as excluding the possibility that one or more other features, numbers, steps, operations, components, ingredients, parts or combinations thereof may be present or added.

In the present specification, the terms “consist of,” “consisting of” or the like are intended to emphasize that there is no other constitution than the features, numbers, steps, operations, components, parts, or combinations thereof described in the specification, and are to be understood as excluding the possibility that one or more other features, numbers, steps, operations, components, parts, or combinations thereof than the above constitution may be present or added.

In the present specification, the term “main ingredient” may refer to an ingredient included in the composition in the largest proportion among all ingredients in the composition according to the present invention. For example, “the first phase ingredient contains Quercus acutissima sap as a main ingredient” may mean that Quercus acutissima sap is contained in the largest proportion among the components of the first phase ingredient.

The present invention relates to a composition for Y-zone cleanliness, a mist including the same, and a method for preparing the composition for Y-zone cleanliness.

The composition for Y-zone cleanliness according to the present invention may be a mixture of a first phase ingredient (A) containing a Quercus acutissima ingredient, specifically Quercus acutissima sap and an ethyl acetate fraction of high-pressure and ultrasonic extract of Quercus acutissima bark and a second ingredient (A) containing zinc ricinoleate, so as to provide antibacterial and deodorizing effects and alleviate acne and atopic dermatitis or provide a skin-calming function.

In general, the ingredients obtained or extracted from Quercus acutissima may be present in various ways, such as leaf extracts, sap, bark extracts or the like. However, there is no example of using an ingredient obtained from Quercus acutissima in a composition providing an antibacterial action for female Y-zone cleanliness and an acne and atopy-alleviating effect.

In addition, the ingredients obtained from Quercus acutissima have a marked difference in the effects derived depending on a specific kind of the ingredients or an extraction method and a purification method, and thus it is required to select an appropriate combination of ingredients, an extraction method and a purification method.

Furthermore, from a viewpoint of the stability of the composition through uniform mixing of the phase of the ingredients obtained from Quercus acutissima and the phase of the ingredients providing a deodorizing function in the Y-zone cleaning agent for women, the type of ingredient obtained from Quercus acutissima, and selection and combination of extraction and purification methods are very important.

As a result of repeated research for many years, the present inventors have confirmed that the use of Quercus acutissima ingredients, specifically Quercus acutissima sap purified with high purity by a specific process and an ethyl acetate fraction of high-pressure and ultrasonic extract of Quercus acutissima bark together with a natural preservative ingredient, maximizes an antibacterial effect and an acne and atopy-alleviating effect, and have also confirmed that the mixing with the phase that provides deodorizing and moisturizing effects at an appropriate ratio leads to a uniform phase composition capable of securing skin-calming or deodorizing effects as well as antibacterial and acne and atopy-alleviating effects, thereby completing the present invention.

The composition for Y-zone cleanliness according to the present invention may include, in particular, Quercus acutissima sap prepared from a process of producing Quercus acutissima sap with minimal mixing of impurities therein, and also include an ethyl acetate fraction of high-pressure and ultrasonic extract of Quercus acutissima bark prepared from an extraction and fractionation process capable of maximizing the extraction of functional effective ingredients that provide antibacterial and acne and atopy-alleviating effects, so that the composition contains a naturally derived ingredient as a main ingredient without being mixed with impurities and thus is harmless to the human body, and inhibits the growth of Staphylococcus epidermidis strain, a secondary infection bacillus, as well as a first infection bacillus, and also inhibits the growth of Candida albicans (ATCC 10231), a yeast that causes inflammatory or bacterial skin diseases, so as to provide very excellent antibacterial and acne and atopy-alleviating effects.

The composition for Y-zone cleanliness according to the present invention may include a mixture of a first phase ingredient (A) containing Quercus acutissima ingredients and a second phase ingredient (B) providing a deodorizing function. In the present specification, the distinction between the first phase ingredient (A) and the second phase ingredient (B) is aimed at specifying each individually prepared ingredient group during the preparation of the composition, and does not mean that the phases of respective ingredients are physically or chemically distinguished from each other in the mixed composition. Meanwhile, the first phase ingredient (A) and the second phase ingredient (B) may be each individually a water phase, an oil phase, or an emulsion phase before mixing, and specifically the first phase ingredient (A) may be a water phase and the second phase ingredient (B) may be an emulsion phase.

The composition for Y-zone cleanliness may include 70 to 90 wt % of the first phase ingredient (A) and 10 to 30 wt % of the second phase ingredient (B). In other words, the composition for Y-zone cleanliness may contain the first phase ingredient (A) as a main ingredient and contain the second phase ingredient (B) as an auxiliary ingredient.

The first phase ingredient (A) may include 40 to 60 parts by weight of Quercus acutissima sap, 20 to 40 parts by weight of an ethyl acetate fraction of high-pressure and ultrasonic extract of Quercus acutissima bark, 0.05 to 2 parts by weight of water-phase zinc gluconate, 0.1 to 3 parts by weight of Zanthoxylum piperitum fruit extract, 0.1 to 3 parts by weight of Pulsatilla koreana flower extract, and 0.1 to 3 parts by weight of Usnea barbata extract.

The Quercus acutissima sap included in the first ingredient (A) may not only serve as a solvent, but also provide an antibacterial activity against Staphylococcus epidermidis, which is a secondary skin infection bacillus after infection with acne germs, and may inhibit the growth of Candida albicans (ATCC 10231), a yeast that causes inflammatory or bacterial skin diseases, so as to provide an antibacterial property and an acne and atopy-alleviating property.

General Quercus acutissima sap may be prepared by cutting Quercus acutissima wood, performing an ultrasonic treatment, etc. to extract the sap, and filtering the same through activated carbon, a filter or the like. In this case, impurities may be mixed in the Quercus acutissima sap, thereby causing a problem of lowering the purity of Quercus acutissima sap. However, the Quercus acutissima sap included in the composition of the present invention may be a condensed water prepared by producing Quercus acutissima sap vapor in a drying furnace of which an outside is captured by a steam tank, and condensing the same in a cooling tank, thereby providing an advantage of accomplishing high purity without mixing of impurities, and this Quercus acutissima sap with high purity may contribute to improving an antibacterial effect of the composition and providing whitening functionality.

Specifically, the Quercus acutissima sap according to the present invention may be a main ingredient accounting for the highest proportion among the ingredients contained in the composition as well as the first phase ingredient (A), and may be prepared through the steps of putting Quercus acutissima wood chips into a drying furnace of which an outside is captured by a steam tank and drying to produce Quercus acutissima sap vapor, and collecting condensed water produced by bringing the Quercus acutissima sap vapor into contact with a cooling coil in a cooling tank.

The Quercus acutissima sap according to the present invention may be prepared by using a sap extracting device disclosed in Korean Patent Registration Publication No. 10-2072667, but is not limited thereto, and any method may be used in the present invention without limitation as long as the method is to obtain Quercus acutissima sap through the condensation of water vapor generated according to the drying of Quercus acutissima wood.

The Quercus acutissima sap may be included in the first phase ingredient (A) as a main ingredient within the range of 40 to 60 parts by weight. In another example, the Quercus acutissima sap may be included in the first phase ingredient (A) within the range of 50 to 60 parts by weight or 55 to 60 parts by weight. In the present specification, the term “part by weight” may mean a weight ratio between respective ingredients unless otherwise indicated.

The ethyl acetate fraction of high-pressure and ultrasonic extract of Quercus acutissima bark included in the first phase ingredient (A) may be so configured that the fraction contributes to increasing an inhibitory activity against the growth of Staphylococcus epidermidis, which is a secondary skin infection bacillus after infection with acne germs, and also inhibits the growth of Candida albicans (ATCC 10231), a yeast that causes bacterial skin diseases, so as to provide the composition with an antibacterial property and acne and atopy-alleviating properties, and thus may be obtained by a method of producing an ethyl acetate fraction of high-pressure and ultrasonic extract, which is an extraction method of effectively extracting biologically active ingredients from Quercus acutissima bark.

There are various methods for carrying out an extraction with respect to Quercus acutissima bark, such as organic solvent extraction, hot water extraction, ultrasonic extraction or the like. The present inventors have confirmed through various experiments that the composition has the maximized antibacterial property and acne and atopy-alleviating properties by including an ethyl acetate fraction of high-pressure and ultrasonic extract of Quercus acutissima bark obtained by carrying out a high-pressure and ultrasonic extraction with respect to Quercus acutissima bark, performing a hot water extraction and carrying out a solvent fractionation with ethyl acetate in the first phase ingredient (A) along with Quercus acutissima sap, and including the resulting product in the composition at a ratio of 70 to 90 wt %.

An ethyl acetate fraction of high-pressure and ultrasonic extract of Quercus acutissima bark may be included in the first phase ingredient (A) within the range of 20 to 40 parts by weight. In another example, the ethyl acetate fraction of high-pressure and ultrasonic extract of Quercus acutissima bark may be included in the first phase ingredient (A) within the range of 25 to 35 parts by weight or 30 to 35 parts by weight.

Meanwhile, the first phase ingredient (A) may include Quercus acutissima sap more than the ethyl acetate fraction of high-pressure and ultrasonic extract of Quercus acutissima bark. If the first phase ingredient (A) includes Quercus acutissima sap less than the ethyl acetate fraction of high-pressure and ultrasonic extract of Quercus acutissima bark, it is undesirable as the composition may not have the maximized antibacterial property and acne and atopy-alleviating properties, and may not be miscible with other materials along with viscosity or physical properties.

In one specific example, the first phase ingredient (A) may include 50 to 60 parts by weight of Quercus acutissima sap; and 25 to 35 parts by weight of the ethyl acetate fraction of high-pressure and ultrasonic extract of Quercus acutissima bark.

The ethyl acetate fraction of high-pressure and ultrasonic extract of Quercus acutissima bark may be prepared, for example, through the steps of: washing and pulverizing Quercus acutissima bark, followed by drying; preparing a sample by mixing the dried Quercus acutissima bark with distilled water, and carrying out a low-temperature and high-pressure extraction with respect to the sample for 5 to 15 minutes at a low temperature of 20° C. to 30° C. and under a pressure of 4,000 bar to 6,000 bar by using a high-pressure extraction device; carrying out an ultrasonic extraction with respect to the sample that has been subject to the low-temperature and high-pressure extraction for 30 to 90 minutes at a frequency of 40 kHz to 70 kHz by using an ultrasonic extractor; carrying out a hot water extraction with respect to the sample that has been subject to the ultrasonic extraction with distilled water for 10 to 20 hours at a temperature of 40° C. to 60° C. so as to obtain a high-pressure and ultrasonic extract of Quercus acutissima bark; filtering and concentrating the high-pressure and ultrasonic extract, followed by free-drying and pulverization; and dispersing the resulting powdered high-pressure and ultrasonic extract in distilled water, and then carrying out a solvent fractionation using ethyl acetate so as to obtain an ethyl acetate fraction of high-pressure and ultrasonic extract of Quercus acutissima bark.

As described above, in the case of sequentially carrying out low-temperature, high-pressure and ultrasonic extractions with respect to Quercus acutissima bark, an antibacterial effective ingredient may be effectively extracted from Quercus acutissima bark. In the case of carrying out a solvent fractionation with respect to the resulting product by using ethyl acetate, it is possible to stably obtain the ethyl acetate fraction of high-pressure and ultrasonic extract of Quercus acutissima bark with an excellent inhibitory effect on the growth of Staphylococcus epidermidis strain and an excellent suppressive effect on the growth of Candida albicans (ATCC 10231).

The first phase ingredient (A) may also include water-phase zinc gluconate. The water-phase zinc gluconate may be included in the first phase ingredient (A) within the range of 0.05 to 2 parts by weight as an ingredient that enhances an antibacterial effect and serves as a preservative and a skin conditioning agent.

In another example, the water-phase zinc gluconate may be included in the first phase ingredient (A) within the range of 0.1 to 1.5 parts by weight or 0.15 to 1.5 parts by weight.

Meanwhile, general zinc gluconate may be in the form of white granules or crystalline powder, but the water-phase zinc gluconate contained in the first phase ingredient (A) according to the present invention may be water-soluble zinc gluconate solubilized in purified water through a special method, may contain purified water as a main ingredient, and may include a zinc salt of gluconic acid represented by formula 1 below within the range of 50,000 ppm to 100,000 ppm.

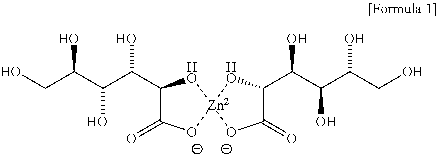

In the above, “containing purified water as a main ingredient” may mean containing purified water in the largest proportion among the constituent ingredients of water-phase zinc gluconate. Specifically, the water-phase zinc gluconate may contain purified water in an amount of at least 50 wt %, at least 60 wt %, at least 70 wt %, at least 80 wt % or at least 90 wt %, and an upper limit of the purified water content may be, for example, 98 wt % or less, 97 wt % or less, or 95 wt % or less.

In addition, the first phase ingredient (A) may further include an ingredient that enhances an antibacterial effect together with the water-phase zinc gluconate.

In one example, the first phase ingredient (A) may further include 0.05 to 1 part by weight of water-phase copper gluconate and 0.05 to 1 part by weight of water-phase magnesium gluconate. The water-phase copper gluconate and water-phase magnesium gluconate may be water-soluble ingredients, each containing a copper salt of gluconic acid and a magnesium salt of gluconic acid within the range of 50,000 ppm to 100,000 ppm.

The first phase ingredient (A) may include each of the Zanthoxylum piperitum fruit extract, Pulsatilla koreana flower extract and Usnea barbata extract within the range of 0.1 to 3 parts by weight, respectively as a natural preservative.

The first phase ingredient (A) may use an ingredient that has good skin affinity and is eco-friendly and harmless to the human body as a preservative so as to further improve an effect of preventing and treating various skin diseases such as acne, atopic dermatitis, etc.

Meanwhile, considering the functions and roles of a natural preservative, it is preferable to use the Zanthoxylum piperitum fruit extract, the Pulsatilla koreana flower extract, and the Usnea barbata extract included in the first phase ingredient (A) from an extraction method capable of minimizing cell destruction or deformation of effective ingredients and effectively extracting effective ingredients, specifically, from a low-temperature water and ultrasonic extraction method.

In one example, the Zanthoxylum piperitum fruit extract, the Pulsatilla koreana flower extract, and the Usnea barbata extract may each include an ultrasonic and low temperature water extract prepared by performing an ultrasonic pretreatment with respect to Zanthoxylum piperitum fruit, Pulsatilla koreana flower and gingko leaves by using an ultrasonic extractor for 30 to 60 minutes under a condition of 100 kHz to 200 kHz, and then carrying out a low-temperature water extraction in a temperature range of 20° C. to 40° C. for 6 to 24 hours.

The first phase ingredient (A) may further include a viscosity controlling agent.

Specifically, the first phase ingredient (A) may further include 0.5 to 5 parts by weight of the viscosity controlling agent.

The viscosity controlling agent may be, for example, at least one selected from butylene glycol, hexylene glycol, ethoxydiglycol, and dipropylene glycol, but is not limited thereto.

In addition, the first phase ingredient (A) may further include purified water in addition to the above-mentioned ingredients. Purified water may be included in the first phase ingredient (A) within the range of, for example, 0.1 to 5 parts by weight or 0.1 to 3 parts by weight.

The first phase ingredient (A) may be included in the composition within the range of 70 to 90 wt %. More specifically, the first phase ingredient (A) may be included in the composition within the range of 75 to 90 wt % or 80 to 90 wt %. If the first phase ingredient (A) is included in an amount of less than 70 wt %, it is natural that the antibacterial and acne and atopy-alleviating effects of the composition are lowered, and the phase stability of the composition may be decreased according to mixing with the second phase ingredient (B). At last, it is undesirable as the uniformity of spraying may be decreased in the mist-type product finally prepared. In addition, if the first phase ingredient (A) is included in the composition in the range of more than 90 wt %, it is also undesirable as a problem with a decrease in the deodorizing effect may occur.

The composition for Y-zone cleanliness according to the present invention may be prepared by mixing the second phase ingredient (B) with the first phase ingredient (A).

The second phase ingredient (B) may include an ingredient that can provide a deodorizing effect of the composition for Y-zone cleanliness and improve a skin-calming effect.

Specifically, the second phase ingredient (B) may include 40 to 60 parts by weight of zinc ricinoleate, 30 to 40 parts by weight of laureth-3, 10 to 20 parts by weight of tetrahydroxypropylethylenediamine, and 1 to 10 parts by weight of propylene glycol.

The zinc ricinoleate included in the second phase ingredient (B) may be a deodorant ingredient or a deodorant, which provides a deodorizing effect to the composition for Y-zone cleanliness.

The zinc ricinoleate included in the second phase ingredient (B) may be included in the second phase ingredient (B) within the range of 40 to 60 parts by weight as a main ingredient of the second phase ingredient (B).

In another example, zinc ricinoleate may be included in the second phase ingredient (B) within the range of 45 to 60 parts by weight or 50 to 60 parts by weight.

The laureth-3 included in the second phase ingredient (B) may be an emulsifier and may be polyethylene glycol ether of lauryl alcohol represented by molecular structural formula 1 below, in which n may be an average of 3.

CH₃(CH₂)₁₁(OCH₂CH₂)ₙOH [Molecular structural formula 1]

Specifically, laureth-3 may be a vegetable ingredient derived from lauryl alcohol of coconut oil, and may serve to help stabilize emulsion by adsorbing to an interface of the constituent material of the second phase ingredient (B).

The laureth-3 may be included in the second phase ingredient (B) within the range of 30 to 40 parts by weight. In another example, the laureth-3 may be included in the second phase ingredient (B) within the range of 30 to 38 parts by weight or 30 to 35 parts by weight.

The tetrahydroxypropylethylenediamine included in the second phase ingredient (B) may be included in the second phase ingredient (B) within the range of 10 to 20 parts by weight as a component serving as a metal chelating agent. In another example, tetrahydroxypropylethylenediamine may be included in the second phase ingredient (B) within the range of 12 to 20 parts by weight or 14 to 20 parts by weight.

The propylene glycol included in the second phase ingredient (B) may be included as a wetting agent in the second phase ingredient (B) within the range of 1 to 10 parts by weight. In another example, propylene glycol may be included in the second phase ingredient (B) within the range of 1 to 8 parts by weight or 1 to 5 parts by weight.

The second phase ingredient (B) may further include a moisturizing enhancer to enhance the skin-calming effect of the composition for Y-zone cleanliness.

In one example, the second phase ingredient (B) may further include panthenol within the range of 0.5 to 2 parts by weight.

The second phase ingredient (B) may be included in the composition as an auxiliary ingredient within the range of 10 to 30 wt %. More specifically, the second phase ingredient (B) may be included in the composition within the range of 10 to 25 wt % or 10 to 20 wt %. If the second phase ingredient (B) is included in an amount of less than 10 wt %, it is undesirable as a problem with a decrease in the deodorizing effect may occur. If the second phase ingredient (B) is included in the composition within the range of more than 30 wt %, it is natural that the antibacterial and acne and atopy-alleviating effects are lowered, and the phase stability of the composition may be decreased according to mixing with the second phase ingredient (B). At last, it is undesirable as the uniformity of spraying may be decreased in the mist-type product finally prepared.

The composition for Y-zone cleanliness according to the present invention may be prepared in the formulation of, for example, solution, cream, emulsion, emollient water, lotion, gel, essence, foundation, pack or stick.

In one example, the composition for Y-zone cleanliness according to the present invention may be contained in a mist for Y-zone cleanliness.

The present invention also relates to a method for preparing the composition for Y-zone cleanliness.

The method for preparing the composition for Y-zone cleanliness may include the steps of: preparing a first phase ingredient (A); preparing a second phase ingredient (B); and mixing the first phase ingredient (A) and the second phase ingredient (B) at a mixing ratio of 7:3 to 9:1(A:B).

The step of preparing the first phase ingredient (A) may include the steps of: putting Quercus acutissima wood chips into a drying furnace of which an outside is captured by a steam tank and drying to produce Quercus acutissima sap vapor, and collecting condensed water produced by bringing the Quercus acutissima sap vapor into contact with a cooling coil in a cooling tank; sequentially carrying out a high-pressure and ultrasonic extraction with regard to a sample including the dried Quercus acutissima bark after washing by using a high-pressure extraction device and an ultrasonic extractor, and then carrying out a solvent fractionation with respect to a freeze-dried product of the high-pressure and ultrasonic extract so as to obtain an ethyl acetate fraction of high-pressure and ultrasonic extract of Quercus acutissima bark; and mixing 40 to 60 parts by weight of Quercus acutissima sap, 20 to 40 parts by weight of an ethyl acetate fraction of high-pressure and ultrasonic extract of Quercus acutissima bark, 0.02 to 2 parts by weight of water-phase zinc gluconate, 0.1 to 3 parts by weight of Zanthoxylum piperitum fruit extract, 0.1 to 3 parts by weight of Pulsatilla koreana flower extract, and 0.1 to 3 parts by weight of Usnea barbata extract.

In addition, the step of obtaining an ethyl acetate fraction of high-pressure and ultrasonic extract of Quercus acutissima bark may include, for example, the steps of: washing and pulverizing Quercus acutissima bark, followed by drying; preparing a sample by mixing the dried Quercus acutissima bark with distilled water, and carrying out a low-temperature and high-pressure extraction with respect to the sample for 5 to 15 minutes at a low temperature of 20° C. to 30° C. and under a pressure of 4,000 bar to 6,000 bar by using a high-pressure extraction device; carrying out an ultrasonic extraction with respect to the sample that has been subject to the low-temperature and high-pressure extraction for 30 to 90 minutes at a frequency of 40 kHz to 70 kHz by using an ultrasonic extractor; carrying out a hot water extraction with respect to the sample that has been subject to the ultrasonic extraction with distilled water for 10 to 20 hours at a temperature of 40° C. to 60° C. so as to obtain a high-pressure and ultrasonic extract of Quercus acutissima bark; filtering and concentrating the high-pressure and ultrasonic extract, followed by free-drying and pulverization; and dispersing the resulting powdered high-pressure and ultrasonic extract in distilled water, and then carrying out a solvent fractionation using ethyl acetate so as to obtain an ethyl acetate fraction of high-pressure and ultrasonic extract of Quercus acutissima bark.

The step of preparing the second phase ingredient (B) may include mixing 40 to 60 parts by weight of zinc ricinoleate, 30 to 40 parts by weight of laureth-3, 10 to 20 parts by weight of tetrahydroxypropylethylenediamine, and 1 to 10 parts by weight of propylene glycol.

An order of the steps of preparing the first phase ingredient (A) and the second phase ingredient (B) is not particularly limited. For example, after the first phase ingredient (A) is prepared in advance, the second phase ingredient (B) may be prepared later. Alternatively, the first phase ingredient (A) may be prepared after the second phase ingredient (B) is prepared in advance.

After preparing the first phase ingredient (A) and the second phase ingredient (B), a step of mixing the two phases may be performed, and specifically a step of mixing the first phase ingredient (A) and the second phase ingredient (B) at a mixing ratio of 7:3 to 9:1(A:B) may be performed. The mixing ratio of the first phase ingredient (A) and the second phase ingredient (B) may be 8:2 to 9:1 in another example.

By the preparation method according to the present invention, there may be prepared the composition for Y-zone cleanliness which is skin-friendly and eco-friendly without side effects to the human body, provides an excellent antibacterial action not only against Propionibacterium acnes, an acne-causing pathogen, but also against Staphylococcus epidermidis, which is a secondary skin infection bacillus after infection with acne germs, and inhibits the growth of Candida albicans (ATCC 10231), a yeast that causes inflammatory or bacterial skin diseases, so as to provide antibacterial and acne and atopy-alleviating effects.

Hereinafter, the composition for Y-zone cleanliness according to the present invention and the mist including the same will be described in more detail with reference to Examples and Comparative Examples.

### [Preparation Example 1]—Preparation for Condensed Water of Quercus acutissima Sap Vapor

The condensed water of Quercus acutissima sap vapor was prepared by using a sap extracting device according to Korean Patent Registration Publication No. 10-2072667.

Specifically, after putting the Quercus acutissima wood chips steamed for 10 minutes at a pressure of 25 kg/cm into a chip drying furnace, the high-temperature Quercus acutissima sap vapor produced by a steam tank surrounding the chip drying furnace was brought into contact with a cooling coil in a cooling tank, so as to produce the condensed water of Quercus acutissima sap vapor (Quercus acutissima sap).

### [Preparation Example 2] Preparation for Ethyl Acetate Fraction of High-Pressure and Ultrasonic Extract of Quercus acutissima Bark

The bark was separated from Quercus acutissima by using a bark scraper (skin peeler, HJ1288), washed with water, and pulverized using a blender. The finely pulverized bark was dried at 50° C. and refrigerated at 4° C., after which the resulting product was subject to a high-pressure and ultrasonic extraction so as to obtain a fraction thereof.

Specifically, the dried Quercus acutissima bark was mixed with distilled water at a ratio of 1:10 to prepare a sample, which was then subject to a low-temperature and high-pressure extraction for 10 minutes at a temperature of 25° C. and under a pressure of 5,000 bar by using a high-pressure extraction device (FOOD CIP-70-350-80, Ilshin Autoclave, Daejeon, Korea). After that, the resulting sample was subject to an ultrasonic extraction for 30 minutes under the condition of 60 kHz by using an ultrasonic extractor (AUG-R3-900, ASIA ULTRASONIC, Gyeonggi, Korea) so as to carry out a high-pressure and ultrasonic extraction. Then, a hot water extraction was performed in an extraction flask with a vertical reflux condenser at a temperature of 50° C. for 10 hours by using distilled water equivalent to 10 times the sample, so as to obtain a high-pressure and ultrasonic extract of Quercus acutissima. Then, the high-pressure and ultrasonic extract was filtered by using a reduced pressure filtration pump (KNF Laboport Pressure Pump, ColeParmer, Vernon Hills, Ill., USA) and 20 to 25 μm filter paper of Whatman (Piscataway, N.J., USA), concentrated by using a reduced pressure rotary vacuum evaporator (NN series, EYELA, Rikakikai Co., Tokyo, Japan), and pulverized into powder by using a freeze dryer (PVTFA 10AT, ILSIN, Suwon, Korea). After that, the resulting powdered high-pressure and ultrasonic extract was dispersed in distilled water, and subject to a solvent fractionation using ethyl acetate so as to obtain an ethyl acetate fraction of high-pressure and ultrasonic extract of Quercus acutissima bark. Then, the fractionated sample was filtered, concentrated and freeze-dried in the same way, and then refrigerated.

### [Preparation Example 3]—Preparation for Natural Preservative Ingredients

Zanthoxylum piperitum fruit, Pulsatilla koreana flower and gingko leaves were each subject to a pretreatment of washing and pulverization so as to prepare an individual sample, which was then subject to an ultrasonic extraction for 40 minutes under the condition of 150 kHz by using an ultrasonic extractor (AUG-R3-900, ASIA ULTRASONIC, Gyeonggi, Korea). Then, a low-temperature water extraction was performed in an extraction flask with a vertical reflux condenser at a temperature of 25° C. for 10 hours by using distilled water equivalent to 10 times the sample, so as to prepare the Zanthoxylum piperitum fruit extract, the Pulsatilla koreana flower extract, and the Usnea barbata extract, respectively.

### [Comparative Preparation Example 1]—Preparation for Hot Water Extract of Quercus acutissima Bark

The same Quercus acutissima bark as used in Preparation Example 2 was subject to a hot water extraction for 24 hours at a temperature of 100° C. by using a hot water extractor (TL-6Point(K), Misung Scientific Co., Yangjoo, Korea), so as to obtain a hot water extract of Quercus acutissima bark. Then, the hot water extract of Quercus acutissima bark was pulverized into powder in the same manner as in Preparation Example 2.

### [Comparative Preparation Example 2]—Preparation for Ethanol Extract of Quercus acutissima Bark

The same Quercus acutissima bark as used in Preparation Example 2 was repeatedly subject to a reflux extraction at 80° C. three times for two hours each by using 75% ethanol as an extraction solvent. The resulting extract was pulverized into powder in the same manner as in Preparation Example 2.

### [Comparative Preparation Example 3]—Preparation for Chloroform Fraction of Hot Water Extract of Quercus acutissima Bark

The powder of the hot water extract of Quercus acutissima prepared according to Comparative Preparation Example 1 was dispersed in distilled water and subject to a solvent fractionation using chloroform to obtain a chloroform fraction of hot water extract of Quercus acutissima, and then a fractionated sample was filtered, concentrated and freeze-dried in the same manner, and then refrigerated.

### [Comparative Preparation Example 4]—Preparation for Chloroform Fraction of Ethanol Extract of Quercus acutissima Bark

The powder of the ethanol extract of Quercus acutissima prepared according to Comparative Preparation Example 2 was dispersed in distilled water and subject to a solvent fractionation using chloroform to obtain a chloroform fraction of ethanol extract of Quercus acutissima, and then a fractionated sample was filtered, concentrated and freeze-dried in the same manner, and then refrigerated.

### [Experiment Preparation]—Experimental Strain and Cultivation

In Experimental Example according to the present invention, Propionibacterium acnes (KCTC3314), which is an acne-causing pathogen, Candida albicans (ATCC 10231), which is a yeast that causes inflammatory or bacterial skin diseases, and Staphylococcus epidermidis (KTTC12228), which is gram-positive skin flora, were used. Propionibacterium acnes (KCTC3314) was inoculated into reinforced clostridial (RC) medium, sealed using a Gaspack system in an anaerobic culture tank, and then anaerobically cultured at 37° C. for two days. As an aerobic strain of Candida albicans (ATCC 10231), Sabaurd Dextrose Agar (Difco. USA) was used, inoculated with the bacteria, and cultured in an incubator at 25° C. for 24 hours. As an aerobic strain, Staphylococcus epidermidis (KTTC12228) was inoculated into Mueller-Hinton medium and cultured at 37° C. for 24 hours before use.

### [Experimental Example 1]—Cytotoxicity Experiment

Human acutemonocytic leukemia cells (THP-1) cultured in RPMI-1640 medium without fetal bovine serum (FBS) were cultured in a 96-well microplate at a concentration of 5×104 cells/well, after which samples according to Preparation Examples 1 and 2 and Comparative Preparation Examples 1 to 4 were administered for each concentration and cultured for 24 hours, and then the supernatant was removed and 100 ul of MTT solution was treated at 36° C. for 4 hours according to an MTT (3-(4,5-dimethylthiazol-2-yl)-2,5-diphenyltetrazolium bromide) measurement method. After removing the culture medium and treating with DMSO 50 ul, absorbance was measured at 540 nm with an absorbance measuring instrument (ELISA) and a measured value was calculated in the same manner as in Equation 1 below.

Cytotoxicity(%)=(1−Absorbance of the group with sample added/Absorbance of the group without sample added)×100 [Equation 1]

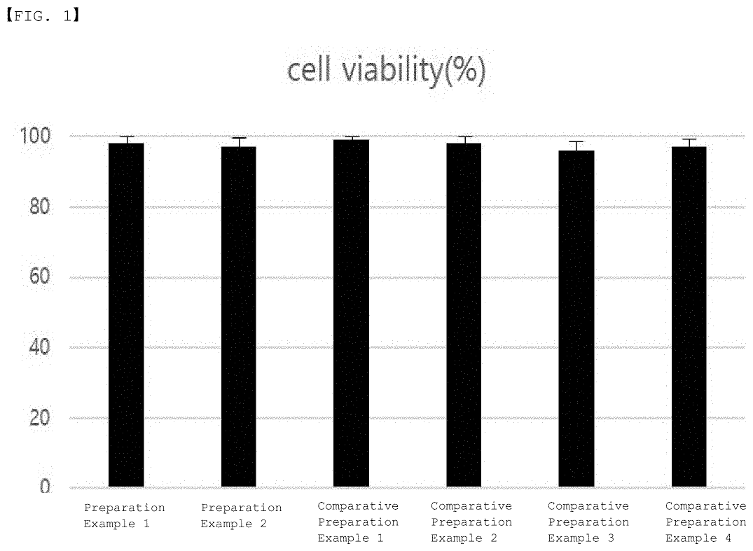

As a result, when the cells were treated with each extract at a concentration of 100 ug/ml, the cell viability was found to be 95% or more, and it was confirmed that the Quercus acutissima sap, the bark extract and the bark fraction according to Preparation Examples 1 and 2 and Comparative Preparation Examples 1 to 4 have no effect on the survival of the cells (see FIG. 1).

[Table 2] Experiment on antibacterial activity against Propionibacterium acnes (KCTC3314) and Candida albicans (ATCC 10231)

With respect to the fraction sample according to Preparation Example 2, antibacterial activity against Propionibacterium acnes (KCTC3314) and Candida albicans (ATCC 10231) was measured in the following manner.

1) Preparation for Bacterial Fluid

An initial culture solution of the test strain was serially diluted 10-fold with phosphate buffer saline (PBS) to prepare 1×10⁶ per ml of the bacterial fluid.

2) Preparation for Test Group and Control Group

The 10 μl of the bacterial fluid was inoculated (1%) into 1 ml of the fraction according to Preparation Example 2, well suspended, and left alone at room temperature for 1 to 15 minutes, so as to prepare the test group. The 10 μl of the bacterial fluid was inoculated (1%) into 1 ml of PBS buffer, well suspended, and left alone at room temperature, so as to prepare the control group.

3) Medium Inoculation of Test Group

The 9 ml of a liquid medium for each strain (Propionibacterium acnes (KCTC3314); RCB, Candida albicans (ATCC 10231); SDB) was put into the test group left alone for each time period (1 min, 3 min, 5 min, 10 min, and 15 min) and subject to vortexing.

Then, 0.1 ml each of the test group and the liquid medium mixture was divided into two or more plate media, dispensed in a Petri dish, and left alone for at least 30 minutes in a 48° C. water bath, after which a bacterial medium RCA and a fungal medium SDA were dispensed in an amount of to 20 ml and evenly mixed. After that, germs (Propionibacterium acnes, KCTC3314) were cultured at 37.5° C.±2.5° C./(2-3 days) and fungi (Candida albicans, ATCC 10231) were cultured at 25° C.±2.5° C./(3-5 days).

4) Medium Inoculation of Control Group

The 9 ml of PBS buffer was put into the control group and subject to vortexing.

Then, 0.1 ml each of the control group was divided into two or more plate media, dispensed in a Petri dish, and left alone for at least 30 minutes in a 48° C. water bath, after which a bacterial medium RCA and a fungal medium SDA were dispensed in an amount of 15 to 20 ml and evenly mixed. After that, germs were cultured at 37.5° C.±2.5° C./(2-3 days) and fungi were cultured at 25°±2.5° C./(3-5 days).

5) Inhibition Rate (%) Calculation and Evaluation

The number of colonies formed in each group was calculated, and the inhibition rate (%) of the test group compared to the control group was calculated by Equation 2 below.

Inhibition rate (%)=[Number of control bacteria(CFU)−Number of test bacteria(CFU)]/Number of control bacteria (CFU)×100 [Equation 2]

6) Results

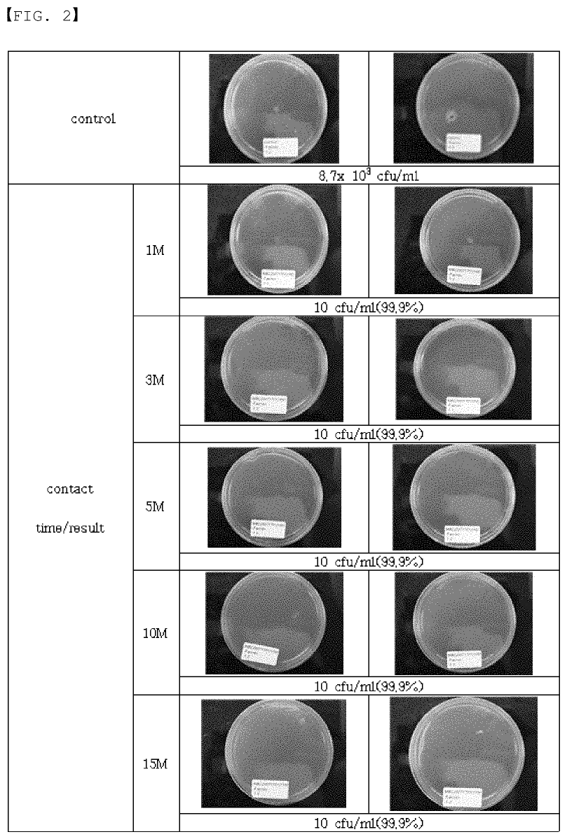

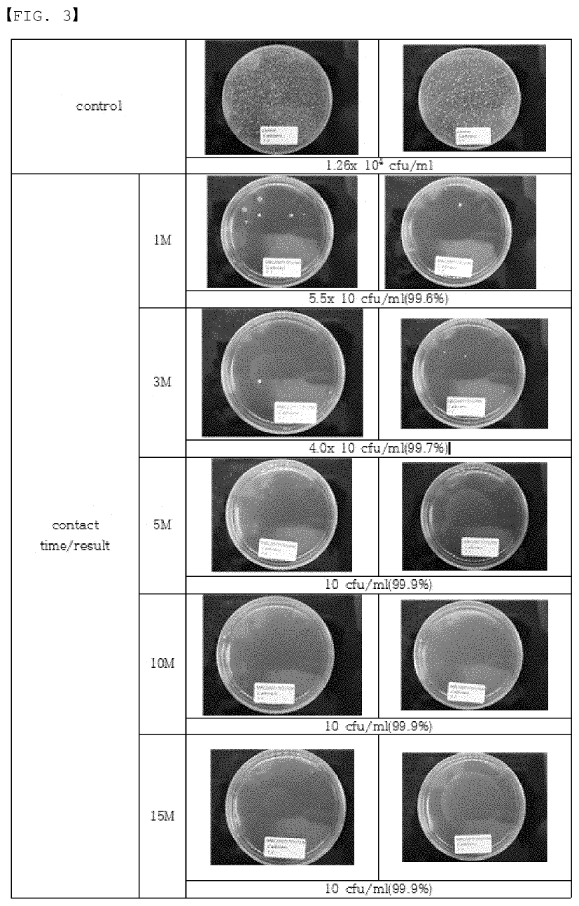

After an experiment on antibacterial activity against Propionibacterium acnes (KCTC3314) and Candida albicans (ATCC 10231) with respect to the fraction according to Preparation Example 2, it was found that the fraction shows a sterilization effect of 99.9% or more on Propionibacterium acnes (KCTC3314), which is anaerobic bacterium, in 1 minute after inoculation of the bacteria and shows a sterilization effect of 99.9% or more on Candida albicans (ATCC 8739), which is yeast, in 5 minutes after inoculation of the bacteria, and thus it could be confirmed that the fraction according to Preparation Example 2 shows an excellent antibacterial property against Propionibacterium acnes (KCTC3314) and Candida albicans (ATCC 10231) (see Table 1, and FIGS. 2 and 3).

TABLE 1
Results of antibacterial activity against
Propionibacterium acnes (KCTC3314) and Candida albicans
(ATCC 10231)

| | Inoculated | | | | | | |
|---|---|---|---|---|---|---|---|
| Test target | Count | | Contact Time/Result (%) | | | | |
| strain | (cfu/ml) | Target | 1M | 3M | 5M | 10M | 15M |
| Propionibacterium acnes | 8.7 × 10³ | Test group | 99.9 | 99.9 | 99.9 | 99.9 | 99.9 |
| (KCTC 3314) | | (Preparation | | | | | |
| | | Example 2 + | | | | | |
| | | Strain) | | | | | |
| Candida Albicans | 1.26 × 10⁴ | Test group | 99.6 | 99.7 | 99.9 | 99.9 | 99.9 |
| (ATCC 10231) | | (Preparation | | | | | |
| | | Example 2 + | | | | | |
| | | Strain) | | | | | |

### [Experimental Example 3]—Experiment on Antibacterial Activity Against Staphylococcus epidermidis (KTTC12228)

With respect to the ingredients according to Preparation Example and Comparative Preparation Example, an antibacterial activity against Staphylococcus epidermidis (KTC12228) was measured by using a paper disk diffusion method.

Specifically, the evaluation of antibacterial activity against Staphylococcus epidermidis (KTTC12228), which is a secondary acne-causing pathogen, was performed by carrying out a cultivation with Muller-Hinton medium at 37° C. for 24 hours, and evenly spreading a bacterial suspension adjusted at a constant concentration (absorbance of 0.5 at an optical density of 620 nm) on an RC agar plate using a sterile cotton swab. After that, the plate was dried and a paper disk (Φ 8 mm, Adventec. USA) was placed on the surface of the plate inoculated with the strain, after which samples according to Preparation Example 2 and Comparative Preparation Examples 1 to 4, the positive control tetracycline and a mixed sample of Preparation Examples 1 and 2 (6:4) were absorbed thereon for each concentration. The sample-treated plate was cultured in an anaerobic culture tank for three days using the Gaspack system, after which the antibacterial activity was measured by the size of the inhibition zone around the disk, and the results thereof are shown in table 2 below, respectively.

TABLE 2
Results of antibacterial activity against
Staphylococcus epidermidis (KTTC12228)

| | Zone of inhibition | |
|---|---|---|
| | (diameter in mm)* | |
| | Concentration | |
| Sample | 5 mg | 1 mg |
| Preparation Example | 15.94 ± 0.21 | 13.03 ± 0.16 |
| 2 | | |
| Comparative | 11.01 ± 0.06 | 9.13 ± 0.24 |
| Preparation Example | | |
| 1 | | |
| Comparative | 10.01± 0.16 | 8.23 ± 0.19 |
| Preparation Example | | |
| 2 | | |
| Comparative | 11.78 ± 0.24 | 9.21 ± 0.14 |
| Preparation Example | | |
| 3 | | |
| Comparative | 10.05 ± 0.15 | 8.36 ± 0.29 |
| Preparation Example | | |
| 4 | | |
| Tetracycline | 14.92 ± 0.23 | 12.82 ± 0.43 |
| Comparative | 16.41 ± 0.17 | 14.53 ± 0.22 |
| Preparation Example | | |
| 1 + 2 (6:4) | | |

*Values are mean ± SD (n = 3) .

As shown in [Table 2], given that the diameter of the inhibition zone is about 14 mm and 12 mm, respectively when being treated with 5 mg and 1 mg of tetracycline, the positive control, it was confirmed that the ethyl acetate fraction of high-pressure and ultrasonic extract of the Quercus acutissima bark according to Preparation Example 2 shows an excellent antibacterial activity against Staphylococcus epidermidis (KTC12228) and shows an excellent antibacterial activity compared to the hot water extract of Quercus acutissima (Comparative Preparation Example 1) and the ethanol extract (Comparative Preparation Example 2) or chloroform fractions thereof. In particular, it was confirmed that the mixed sample obtained by mixing the Quercus acutissima sap according to Preparation Example 1 and the ethyl acetate fraction of high-pressure and ultrasonic extract of Quercus acutissima bark according to Preparation Example 2 at an appropriate ratio shows the best antibacterial activity. Thus, it was verified through an experiment that, when Quercus acutissima sap and the ethyl acetate fraction of high-pressure and ultrasonic extract of Quercus acutissima bark are mixed at an appropriate ratio, it is possible to prepare a composition that is safer than tetracycline, which has limitations in use, such as side effects, the appearance of resistant bacteria and the like, has no side effects on the human body, and has an excellent antibacterial effect against a secondary acne-causing pathogen.

### [Examples 1 to 4]—Preparation for Composition for Y-Zone Cleanliness (A1 to A4)

Quercus acutissima sap according to Preparation Example 1, Quercus acutissima sap according to Preparation Example 2, water-phase zinc gluconate containing about 60,000 ppm of zinc gluconate along with the ethyl acetate fraction of high-pressure and ultrasonic extract of Quercus acutissima bark, natural preservative ingredients according to Preparation Example 4, and the viscosity controlling agent were adjusted to the contents as shown in [Table 3] below, mixed and stirred to prepare a first phase ingredient (A), zinc ricinoleate, laureth-3, tetrahydroxypropylethylenediamine, propylene glycol and panthenol were adjusted to the contents as shown in [Table 3] below, mixed and stirred to prepare a second phase ingredient (B), and then the first phase ingredient (A) and the second phase ingredient (B) were mixed at a weight ratio of 8:2, respectively to prepare compositions for Y-zone cleanliness (A1 to A4) in an amount of 200 mL each.

TABLE 3

| | | Content (wt %) | | | |
|---|---|---|---|---|---|
| | | Example | Example | Example | Example |
| Ingredient | | 1 (A1) | 2 (A2) | 3 (A3) | 4 (A4) |
| First phase | Quercus acutissima sap | 59 | 55 | 58 | 56 |
| ingredient | according to Preparation | | | | |
| (A) | Example 1 | | | | |
| | Fraction according to | 31 | 33 | 32 | 35 |
| | Preparation Example 2 | | | | |
| | Water-phase zinc | 0.1 | 0.15 | 0.2 | 0.5 |
| | gluconate | | | | |
| | Zanthoxylum piperitum | 1.2 | 1.1 | 1.5 | 1.5 |
| | fruit extract | | | | |
| | Pulsatilla koreana | 1.1 | 1.5 | 0.5 | 0.7 |
| | flower extract | | | | |
| | Usnea Barbata extract | 1.5 | 1.4 | 2 | 0.8 |
| | Viscosity controlling | 4 | 4 | 5 | 5 |
| | agent | | | | |
| | Purified water | Residual | Residual | Residual | Residual |
| | Sub-total | 100 | 100 | 100 | 100 |
| Second phase | Zinc ricinoleate | 51 | 50 | 51 | 50 |
| ingredient | Laureth-3 | 30 | 31 | 30 | 32 |
| (B) | Tetrahydroxypropyl- | 15 | 14 | 15 | 15 |
| | ethylenediamine | | | | |
| | Propylene glycol | 3 | 4 | 3 | 2 |
| | Panthenol | 1 | 1 | 1 | 1 |
| | Sub-total | 100 | 100 | 100 | 100 |

### [Comparative Examples 1 to 4]—Preparation for Composition for Y-Zone Cleanliness (B1 to B4)

According to the content of ingredients as shown in table 4 below, the first phase ingredient (A) and the second phase ingredient (B) were mixed at a weight ratio of 8:2, respectively, so as to prepare the compositions for Y-zone cleanliness (B1 to B3) according to Comparative Examples 1 to 3.

In addition, according to the content of ingredients as shown in table 4 below, the first phase ingredient (A) and the second phase ingredient (B) were mixed at a weight ratio of 6:4, respectively, so as to prepare the composition for Y-zone cleanliness (B4) according to Comparative Example 4.

TABLE 4

| | | Content (wt %) | | | |
|---|---|---|---|---|---|
| | | Comparative | Comparative | Comparative | Comparative |
| | | Example | Example | Example | Example |
| Ingredient | | 1 (B1) | 2 (B2) | 3 (B3) | 4 (B4) |
| First | Quercus acutissima sap | 12 | 20 | — | 30 |
| phase | according to Preparation | | | | |
| ingredient | Example 1 | | | | |
| (A) | Fraction according to | — | — | 10 | 15 |
| | Preparation Example 2 | | | | |
| | Water-phase zinc | 0.1 | 0.15 | 0.3 | 0.8 |
| | gluconate | | | | |
| | Zanthoxylum piperitum | 1.5 | 1.1 | 1.5 | 1.4 |
| | fruit extract | | | | |
| | Pulsatilla koreana | 1.4 | 1.5 | 0.5 | 0.6 |
| | flower extract | | | | |
| | Usnea Barbata extract | 1.0 | 1.4 | 2 | 0.9 |
| | Viscosity controlling | 2 | 3 | 1.5 | 2 |
| | agent | | | | |
| | Purified water | Residual | Residual | Residual | Residual |
| | Sub-total | 100 | 100 | 100 | 100 |
| Second | Zinc ricinoleate | 45 | 30 | 25 | 20 |
| phase | Tetrahydroxypropyl- | 10 | 15 | 13 | 15 |
| Ingredient | ethylenediamine | | | | |
| (B) | Propylene glycol | 8 | 6 | 7 | 8 |
| | Laureth-3 | Residual | Residual | Residual | Residual |
| | Panthenol | 2 | 1 | 1 | 1 |
| | Sub-total | 100 | 100 | 100 | 100 |

### [Experimental Example 4]—Experiment on Spraying Force

An experiment on spraying force was performed by putting the composition for Y-zone cleanliness according to Examples 1 to 4 and Comparative Examples 1 to 4 into a spray-type mist container and spraying toward a square transparent glass having a size of 15 cm×15 cm to observe a particle distribution of the sprayed composition. And the experiment on spraying force was performed based on the distribution of the composition particles and whether or not visible droplet clusters were observed on the transparent glass, and the results thereof were classified into grades 1 (very excellent) to grade 5 (very poor) as shown in table 5 below.

TABLE 5

| | Grade of spraying force |
|---|---|
| Target composition | experiment |
| Example 1 | 1 |
| Example 2 | 1 |
| Example 3 | 1 |
| Example 4 | 1 |
| Comparative Example 1 | 2 |
| Comparative Example 2 | 4 |
| Comparative Example 3 | 4 |
| Comparative Example 4 | 5 |

As shown in above table 5, it was confirmed for Examples 1 to 4, in which the first phase ingredient (A) and the second phase ingredient (B) were mixed at an appropriate ratio, that a spraying force is appropriate. However, it was confirmed for Comparative Example 4, in which the second phase ingredient (B) accounted for 40 wt %, that the spraying force is very poor, and it was also confirmed for Comparative Examples 2 and 3 using the second phase ingredient (B) containing more laureth-3 than zinc ricinoleate that the spraying force is somewhat poor.

### [Experimental Example 5]—Sensory Evaluation of Acne and Atopic Dermatitis

A spray-type mist containing the compositions according to Examples 1 to 4 and Comparative Examples 1 to 4 was prepared, and a clinical test was conducted for 30 women in their 30s to 50s for three months. After the spray-type mist was sprayed on the neck and Y zone, a sensory evaluation including an effect of alleviating/relieving acne, atopic dermatitis and itchiness was performed. Based on the visual and tactile senses, an average value of 30 people was derived by evaluating five levels of very satisfied (5)/satisfactory (4)/average (3)/poor (2)/very poor (1), and the results thereof are shown in table 6 below.

TABLE 6

| | | Atopic | |
|---|---|---|---|
| Target composition | Acne | dermatitis | Itchiness |
| Example 1 | 5 | 5 | 5 |
| Example 2 | 5 | 5 | 4.5 |
| Example 3 | 5 | 4.5 | 5 |
| Example 4 | 4.5 | 5 | 5 |
| Comparative Example 1 | 3 | 2 | 2 |
| Comparative Example 2 | 3 | 1 | 2 |
| Comparative Example 3 | 1 | 1 | 1 |
| Comparative Example 4 | 3 | 3 | 2 |

As shown in above table 6, it was shown that the compositions for Y-zone cleanliness according to Examples 1 to 4 show an excellent effect of alleviating acne and atopic dermatitis and reducing itchiness, but it was confirmed that the compositions according to Comparative Examples 1 to 4 show a less excellent or very insufficient effect than those of Examples.

### [Experimental Example 6]—Experiment on Skin-Calming Effect

For an experimental group according to above Experimental Example 5, in order to provide the same effect as when a face temperature rises in summer, subjects were asked to jump rope for 20 minutes to generate heat on the face, after which the compositions according to Examples 1 to 4 were sprayed to sheets. The sheets were placed on the cheekbones of the subject and left alone for 20 minutes, after which the sample was removed, and a skin temperature was measured over time from immediately after removal to conduct an experiment on the skin-calming effect, and the results thereof are shown in table 7 below.

TABLE 7

| Target | After | After sample | After 10 | After 20 |
|---|---|---|---|---|
| composition | exercise | removal | min | min |
| Example 1 | 33.9° C. | 31.5° C. | 31.2° C. | 31.3° C. |
| Example 2 | 33.5° C. | 31.2° C. | 31.2° C. | 31.3° C. |
| Example 3 | 33.7° C. | 31.5° C. | 31.4° C. | 31.2° C. |
| Example 4 | 33.7° C. | 31.2° C. | 31.3° C. | 31.1° C. |

As shown in above table 7, it can be confirmed that a skin temperature is rapidly lowered as the sheets sprayed with the compositions according to Examples 1 to 4 are applied to the skin, and thus it could be confirmed that the composition according to the present invention has an excellent skin-calming effect.

### [Experimental Example 7]—Experiment on Skin-Calming Effect

A deodorizing effect of the compositions according to Examples 1 to 4 was evaluated based on a concentration reduction rate of ammonia or trimethylamine.

Specifically, each 20 mL of the samples according to Examples 1 to 4 and Comparative Examples 1 to 4 was put into a 5 L reactor and sealed, and then ammonia or trimethylamine was injected as a test gas at an initial concentration of 50 umol/mol, and the concentration of the test gas was measured at the time of initial (0 min), 30 min, 60 min, 90 min, and 120 min (test group), and a concentration reduction rate of the test gas for each time period was measured in comparison with the control group without the composition. A specific equation is the same as in equation 1 below, and the results thereof are shown in tables 8 and 9 below.

Test gas concentration reduction(%)=(Control group concentration−Test group concentration)/Control group concentration×100 [Equation 1]

TABLE 8

| | Ammonia concentration reduction (%) | | | |
|---|---|---|---|---|
| Time | Example 1 | Example 2 | Example 3 | Example 4 |
| 0 min | 0 | 0 | 0 | 0 |
| 30 min | 99.7 | 99.6 | 99.7 | 99.5 |
| 60 min | 99.7 | 99.6 | 99.7 | 99.5 |
| 90 min | 99.7 | 99.6 | 99.7 | 99.5 |
| 120 min | 99.7 | 99.6 | 99.7 | 99.5 |

TABLE 9

| | Trimethylamine concentration reduction (%) | | | |
|---|---|---|---|---|
| Time | Example 1 | Example 2 | Example 3 | Example 4 |
| 0 min | 0 | 0 | 0 | 0 |
| 30 min | 98 | 97 | 97.5 | 97.2 |
| 60 min | 98.5 | 96.5 | 98.2 | 98.1 |
| 90 min | 98.7 | 97.2 | 99.0 | 98.2 |
| 120 min | 98.9 | 98.5 | 99.0 | 98.8 |

As shown in above tables 8 and 9, it was confirmed that the compositions for Y-zone cleanliness according to Examples 1 to 4 have an excellent deodorizing effect.

What is claimed is:

1. A composition for Y-zone cleanliness, the composition comprising a mixture of:
70 to 90 wt % of a first phase ingredient (A) including 40 to 60 parts by weight of Quercus acutissima sap, 20 to 40 parts by weight of an ethyl acetate fraction of high-pressure and ultrasonic extract of Quercus acutissima bark, 0.05 to 2 parts by weight of water-phase zinc gluconate containing a zinc salt of gluconic acid represented by formula 1 below within a range of 50,000 ppm to 100,000 ppm as water-soluble zinc gluconate solubilized in purified water, 0.1 to 3 parts by weight of Zanthoxylum piperitum fruit extract, 0.1 to 3 parts by weight of Pulsatilla koreana flower extract, and 0.1 to 3 parts by weight of Usnea barbata extract; and
10 to 30 wt % of a second phase ingredient (B) including 40 to 60 parts by weight of zinc ricinoleate, 30 to 40 parts by weight of laureth-3, 10 to 20 parts by weight of tetrahydroxypropylethylenediamine, and 1 to 10 parts by weight of propylene glycol,
wherein the ethyl acetate fraction of high-pressure and ultrasonic extract of Quercus acutissima bark is prepared through the steps of:
washing and pulverizing Quercus acutissima bark, followed by drying;
preparing a sample by mixing the dried Quercus acutissima bark with distilled water, and carrying out a low-temperature and high-pressure extraction with respect to the sample for 5 to 15 minutes at a low temperature of 20° C. to 30° C. and under a pressure of 4,000 bar to 6,000 bar by using a high-pressure extraction device;
carrying out an ultrasonic extraction with respect to the sample that has been subject to the low-temperature and high-pressure extraction for 30 to 90 minutes at a frequency of 40 kHz to 70 kHz by using an ultrasonic extractor;
carrying out a hot water extraction with respect to the sample that has been subject to the ultrasonic extraction with distilled water for 10 to 20 hours at a temperature of 40° C. to 60° C. so as to obtain a high-pressure and ultrasonic extract of Quercus acutissima bark;
filtering and concentrating the high-pressure and ultrasonic extract, followed by freeze-drying and pulverization; and
dispersing the resulting powdered high-pressure and ultrasonic extract in distilled water, and then carrying out a solvent fractionation using ethyl acetate so as to obtain an ethyl acetate fraction of high-pressure and ultrasonic extract of Quercus acutissima bark.

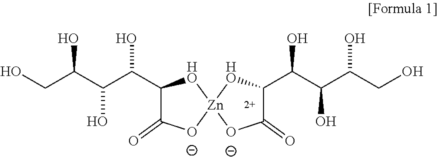

2. The composition of claim 1, wherein the Quercus acutissima sap is prepared through the steps of putting Quercus acutissima wood chips into a drying furnace of which an outside is captured by a steam tank and drying to produce Quercus acutissima sap vapor, and bringing the Quercus acutissima sap vapor into contact with a cooling coil in a cooling tank, thereby collecting condensed water produced.

3. The composition of claim 1, wherein the first phase ingredient (A) comprises:
50 to 60 parts by weight of Quercus acutissima sap; and
25 to 35 parts by weight of the ethyl acetate fraction of high-pressure and ultrasonic extract of Quercus acutissima bark.

4. The composition of claim 1, wherein the Zanthoxylum piperitum fruit extract, the Pulsatilla koreana flower extract, and the Usnea barbata extract each comprises an ultrasonic and low temperature water extract prepared by performing an ultrasonic pretreatment with respect to Zanthoxylum piperitum fruit, Pulsatilla koreana flower and gingko leaves by using an ultrasonic extractor for 30 to 60 minutes under a condition of 100 kHz to 200 kHz, and then carrying out a low-temperature water extraction in a temperature range of 20° C. to 40° C. for 6 to 24 hours.

5. The composition of claim 1, wherein the second phase ingredient (B) further comprises:
0.5 to 2 parts by weight of panthenol.

6. A mist for Y-zone cleanliness comprising the composition for Y-zone cleanliness of claim 1.
